(19) United States
(12) Patent Application Publication
(10) Pub. No.: US 2019/0231319 A1
(43) Pub. Date: Aug. 1, 2019

(54) ULTRASONIC SIGNAL PROCESSING DEVICE, ULTRASONIC SIGNAL PROCESSING METHOD, AND ULTRASONIC DIAGNOSTIC DEVICE

(71) Applicant: Konica Minolta, Inc., Tokyo (JP)

(72) Inventor: Yasuaki SUSUMU, Osaka (JP)

(21) Appl. No.: 16/248,979

(22) Filed: Jan. 16, 2019

(30) Foreign Application Priority Data
Jan. 31, 2018 (JP) 2018-015216

(51) Int. Cl. A61B 8/08; A61B 8/00

(52) U.S. Cl. CPC A61B 8/5207; A61B 8/06; A61B 8/4488; A61B 8/488

(57) ABSTRACT

An ultrasonic signal processing device that performs speed analysis by a color flow mapping method by executing ultrasonic transmission/reception to/from a subject by driving a plurality of transducers arranged in array on an ultrasonic probe, includes: a transmitter that repeatedly executes a process of selecting two or more transmission conditions in predetermined order and transmitting an ultrasonic wave prescribed under the selected transmission condition into the subject; a reception beam former that generates an acoustic line signal on the basis of a reflected ultrasonic wave in synchronization with the ultrasonic transmission by the transmitter; a quadrature detector that performs quadrature detection on the acoustic line signal to generate a complex acoustic line signal; and a speed calculator that performs a process of grouping a plurality of complex acoustic line signals as a packet for each transmission condition and analyzes per packet to generate speed information in the subject.

The entire disclosure of Japanese patent Application No. 2018-015216, filed on Jan. 31, 2018, is incorporated herein by reference in its entirety.

### BACKGROUND

### Technological Field

The present disclosure relates to an ultrasonic signal processing device, an ultrasonic signal processing method, and an ultrasonic diagnostic device provided with the same, and especially relates to an ultrasonic transmitting/receiving method in an ultrasonic signal processing device using a color flow mapping method, and color flow mapping arithmetic processing.

### Description of the Related Art

The ultrasonic diagnostic device transmits ultrasonic waves into a subject by an ultrasonic probe (hereinafter referred to as a “probe”) and receives ultrasonic reflected waves (echos) generated by a difference in acoustic impedance of subject tissue. Furthermore, on the basis of an electric signal obtained from the reception, an image illustrating a structure of internal tissue of the subject is generated to be displayed on a monitor (hereinafter referred to as a “display unit”). With the ultrasonic diagnostic device which is less invasive to the subject, a state of in-body tissue may be observed in real time by tomographic images and the like, so that this is widely used for morphological diagnosis of a living body.

In recent years, many ultrasonic diagnostic devices are equipped with a color flow mapping (CFM) method. In the CFM method, a Doppler shift (frequency shift) occurring in an echo due to movement of in-body tissue such as a blood flow is detected, and speed information is made a two-dimensional image to be superimposed on a two-dimensional tomographic image (B-mode tomographic image). In order to detect the Doppler shift, it is necessary to repeatedly transmit and receive the ultrasonic waves to the same position in the subject. Hereinafter, a time interval at which the ultrasonic waves are transmitted and received at the same position is referred to as “pulse repetition time”.

In recent years, improvement in detection accuracy with respect to low speed movement such as a fine blood vessel is desired, and the pulse repetition time tends to increase. For this reason, a sequence is used in which ultrasonic scanning is performed at equal intervals with respect to an entire target area in the subject, rather than continuously transmitting ultrasonic waves to the same position in the subject.

In the CFM method, the accuracy of obtained speed information depends on transmission conditions such as a frequency and a transmission direction of the ultrasonic waves, so that optimization of the transmission conditions is desired for improving the accuracy of the CFM method. On the other hand, it is difficult to know the optimum transmission condition in advance, and there is a problem that the optimum transmission condition in a target area is not always constant.

### SUMMARY

The present invention is achieved in view of the above-described problems, and an object thereof is to prevent variation in quality of color Doppler images in the target area and to improve the quality of the color Doppler images of the entire target area.

To achieve the abovementioned object, according to an aspect of the present invention, there is provided an ultrasonic signal processing device that performs speed analysis by a color flow mapping method by executing ultrasonic transmission/reception to/from a subject by driving a plurality of transducers arranged in array on an ultrasonic probe, and the ultrasonic signal processing device reflecting one aspect of the present invention comprises: a transmitter that repeatedly executes a process of selecting two or more transmission conditions in predetermined order and transmitting an ultrasonic wave prescribed under the selected transmission condition into the subject at predetermined time intervals; a reception beam former that generates an acoustic line signal on the basis of a reflected ultrasonic wave received by the transducers in synchronization with the ultrasonic transmission by the transmitter; a quadrature detector that performs quadrature detection on the acoustic line signal to generate a complex acoustic line signal; and a speed calculator that performs a process of grouping a plurality of complex acoustic line signals corresponding to the same transmission condition as a packet for each transmission condition and analyzes per packet to generate speed information in the subject.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a functional block diagram of an ultrasonic diagnostic system according to an embodiment;

FIG. 2 is a functional block diagram illustrating a configuration of a reception beam former according to the embodiment;

FIG. 3A is a schematic diagram illustrating a propagation path of an ultrasonic transmission wave by a transmission beam former according to the embodiment;

FIG. 3B is a schematic diagram illustrating an acoustic line target area by a reception beam former;

FIG. 4 is a functional block diagram illustrating configurations of a CFM processor, a tomographic image processor, and an image generator according to the embodiment;

FIG. 5A is a time chart illustrating a relationship between an execution time of a transmission/reception event and a transmission/reception area of ultrasonic waves according to the embodiment;

FIG. 5B is an example of a transmission condition;

FIGS. 6A and 6B are schematic diagrams illustrating speed synthesis by a speed synthesizing unit according to the embodiment;

FIG. 7 is a flowchart illustrating operation of an ultrasonic diagnostic device according to the embodiment;

FIG. 8 is a flowchart illustrating a CFM process in the CFM processor according to the embodiment;

FIG. 9 is an example of a time chart illustrating a relationship between an execution time of a transmission/reception event and a transmission/reception area of ultrasonic waves according to a first variation;

FIG. 10 is an example of a time chart illustrating a relationship between an execution time of a transmission/reception event and a transmission/reception area of ultrasonic waves according to the first variation;

FIGS. 11A and 11B are schematic diagrams illustrating speed synthesis by a speed synthesizing unit according to a second variation; and

FIG. 12 is an example of a time chart illustrating a conventional relationship between an execution time of a transmission/reception event and a transmission/reception area of ultrasonic waves.

### DETAILED DESCRIPTION OF EMBODIMENTS

Hereinafter, one or more embodiments of the present invention will be described with reference to the drawings. However, the scope of the invention is not limited to the disclosed embodiments.

### Background to Mode for Carrying Out Invention

The inventor conducted various studies for improving detecting accuracy of a speed in an entire target area without decreasing a frame rate in an ultrasonic diagnostic device which generates a color Doppler image.

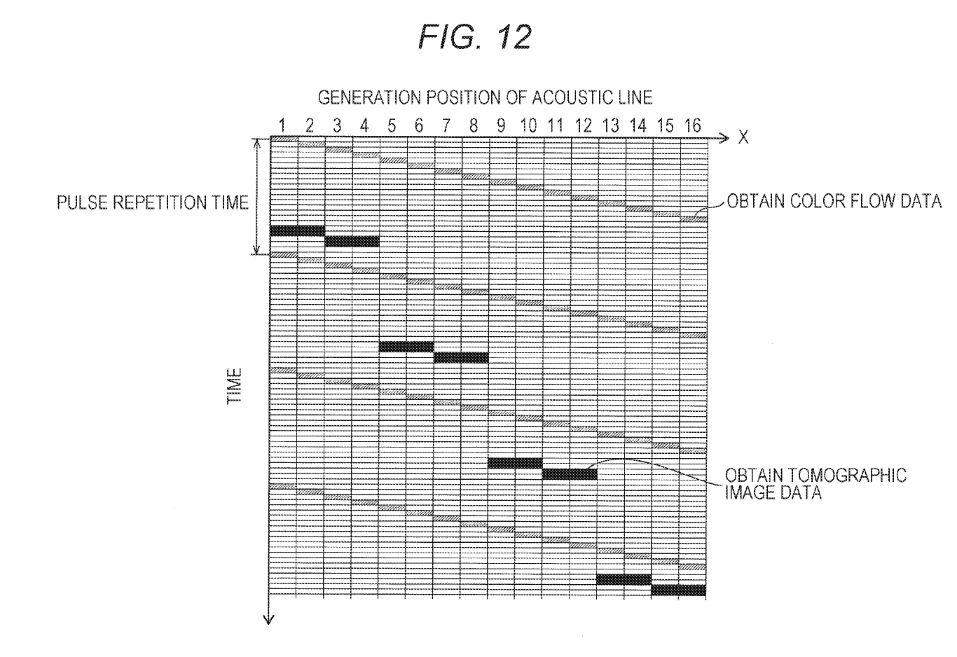

In the ultrasonic diagnostic device performing a CFM method, for example, operation on the basis of a time chart illustrating a relationship between an execution time of a transmission/reception event and a transmission/reception area of ultrasonic waves as illustrated in FIG. 12 is conventionally performed. A position of a transmission focal point F in an element array direction (x direction) and a generation position of an acoustic line are plotted along a horizontal axis (X axis) in FIG. 12. Note that, in FIG. 12, the number of transducers is set to 16 for convenience.

As illustrated in FIG. 12, in the conventional ultrasonic diagnostic device, for example, operation of generating an acoustic line signal for a linear area passing through the transmission focal point in synchronization with ultrasonic transmission, and moving the transmission focal point and the generation position of the acoustic line signal by one transducer each time the ultrasonic transmission/reception is performed, for example. As a result, a pulse repetition time which is an interval of the ultrasonic transmission/reception may be made long at one observation point in a target area. On the other hand, if it is tried to detect a Doppler shift while the acoustic line signals of different transmission conditions are mixed, the detection accuracy of the speed is lowered, so that it is not possible to change the transmission condition in the middle of a series of ultrasonic transmission/reception. Therefore, in order to optimize the transmission condition, it is necessary to perform the CFM method under a first transmission condition and the CFM method under a second transmission condition so as not to interfere with each other. However, when the CFM method under the first transmission condition and the CFM method under the second transmission condition are separately performed, a required time is generated in each case, so that the frame rate decreases in inverse proportion to the number of transmission conditions. Therefore, for example, as disclosed in JP 6104749 B2, there conventionally is a technology of improving an S/N ratio of the acoustic line signal by performing spatial compounding at the time of generation of the acoustic line signal. However, in this technology, only one type of CFM method is performed, and the CFM method under the first transmission condition and the CFM method under the second transmission condition are not separately performed.

Therefore, in view of the above-described problem, the inventor focused on performing the CFM method under the first transmission condition and the CFM method under the second transmission condition so as not to interfere with each other while sharing most of the required time, and achieved an ultrasonic signal processing method and an ultrasonic diagnostic device using the same according to the embodiment.

Hereinafter, the ultrasonic image processing method and the ultrasonic diagnostic device using the same according to the embodiment are described in detail with reference to the drawings.

### Embodiment

### Overall Configuration

Hereinafter, an ultrasonic diagnostic device 100 according to an embodiment is described with reference to the drawings.

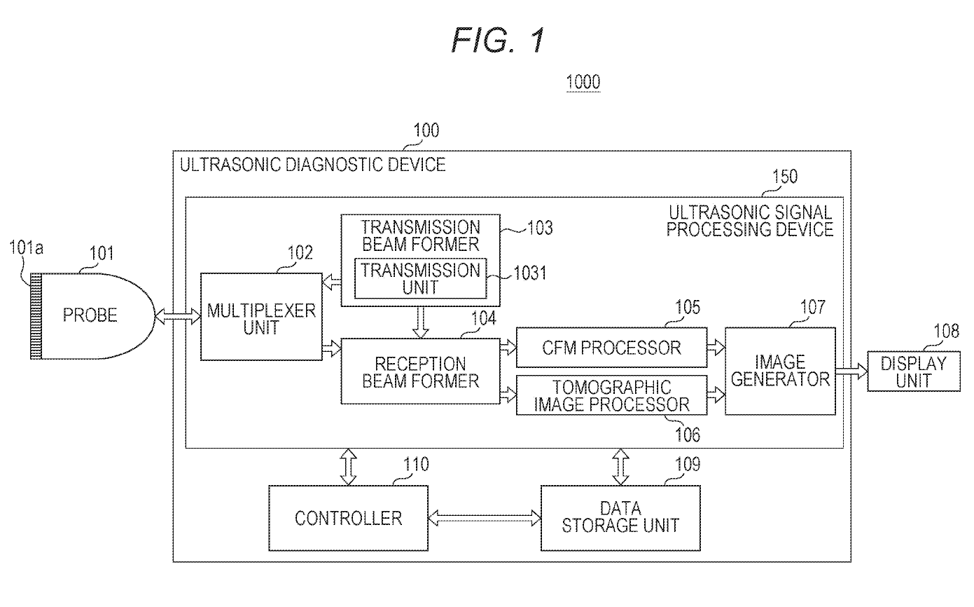

FIG. 1 is a functional block diagram of an ultrasonic diagnostic system 1000 according to the embodiment. As illustrated in FIG. 1, the ultrasonic diagnostic system 1000 includes a probe 101 including a plurality of transducers 101a which transmits ultrasonic waves to a subject and receives reflected waves thereof, the ultrasonic diagnostic device 100 which allows the probe 101 to transmit/receive the ultrasonic waves and generates an ultrasonic image on the basis of an output signal from the probe 101, and a display unit 108 which displays the ultrasonic image on a screen. The probe 101 and the display unit 108 are configured to be connectable to the ultrasonic diagnostic device 100. FIG. 1 illustrates a state in which the probe 101 and the display unit 108 are connected to the ultrasonic diagnostic device 100. Note that the probe 101 and the display unit 108 may be provided inside the ultrasonic diagnostic device 100.

### Configuration of Ultrasonic Diagnostic Device 100

The ultrasonic diagnostic device 100 includes a multiplexer unit 102 which selects a transducer used when transmitting or receiving out of a plurality of transducers 101a of the probe 101 and securing input/output to/from the selected transducer, a transmission beam former 103 which controls timing to apply high voltage to each transducer 101a of the probe 101 in order to transmit the ultrasonic wave, and a reception beam former 104 which amplifies electric signals obtained by a plurality of transducers 101a, A/D converts the same, and performs reception beam forming to generate acoustic line signals on the basis of the reflected waves of the ultrasonic waves received by the probe 101. In addition, a CFM processor 105 which performs frequency analysis on an output signal from the reception beam former 104 to generate color flow information, a tomographic image processor 106 which generates a frame acoustic line signal corresponding to a tomographic image (B-mode image) on the basis of the output signal from the reception beam former 104, an image generator 107 which converts the frame acoustic line signal into the B-mode tomographic image, superimposes the color flow information thereon to generate a color Doppler image, and displays the same on the display unit 108, a data storage unit 109 which stores the acoustic line signal output by the reception beam former 104, a frame CFM signal output by the CFM processor 105, and a frame acoustic line signal output by the tomographic image processor 106, and a controller 110 which controls each component are provided.

Among them, the multiplexer unit 102, the transmission beam former 103, the reception beam former 104, the CFM processor 105, the tomographic image processor 106, and the image generator 107 form the ultrasonic signal processing device 150.

Each of components forming the ultrasonic diagnostic device 100, for example, the multiplexer unit 102, the transmission beam former 103, the reception beam former 104, the CFM processor 105, the tomographic image processor 106, the image generator 107, and the controller 110 is realized by, for example, a hardware circuit such as a field programmable gate array (FPGA) and an application specific integrated circuit (ASIC).

The data storage unit 109 is a computer readable recording medium and, for example, a flexible disk, a hard disk, an MO, a DVD, a DVD-RAM, a BD, a semiconductor memory or the like may be used. The data storage unit 109 may also be a storage device externally connected to the ultrasonic diagnostic device 100.

Note that the ultrasonic diagnostic device 100 according to this embodiment is not limited to the ultrasonic diagnostic device having a configuration illustrated in FIG. 1. For example, it is also possible that there is no multiplexer unit 102, and the transmission beam former 103 and the reception beam former 104 are directly connected to each transducer 101a of the probe 101. Also, the transmission beam former 103, the reception beam former 104, a part thereof and the like may be incorporated in the probe 101. This is not limited to the ultrasonic diagnostic device 100 according to this embodiment, and the same applies to an ultrasonic diagnostic device according to other embodiments and variations to be described later.

### Description of Each Component

1. Transmission Beam Former 103

The transmission beam former 103 is connected to the probe 101 via the multiplexer unit 102 and controls timing to apply high voltage to each of a plurality of transducers included in a transmission opening Tx including a transmission transducer array of all or a part of a plurality of transducers 101a present in the probe 101 for transmitting the ultrasonic waves from the probe 101. The transmission beam former 103 includes a transmitter 1031.

On the basis of a transmission control signal from the controller 110, the transmitter 1031 performs a transmitting process of supplying a pulsed transmission signal for transmitting an ultrasonic beam to each transducer included in the transmission opening Tx out of a plurality of transducers 101a present in the probe 101. Specifically, the transmitter 1031 is provided with, for example, a clock generation circuit, a pulse generation circuit, and a delay circuit. The clock generation circuit is a circuit which generates a clock signal which determines transmission timing of the ultrasonic beam. The pulse generation circuit is a circuit for generating a pulse signal which drives each transducer. The delay circuit is a circuit for setting a delay time of the transmission timing of the ultrasonic beam for each transducer and delaying the transmission of the ultrasonic beam by the delay time to focus the ultrasonic beam.

The transmitter 1031 repeatedly performs ultrasonic transmission while moving the transmission opening Tx by a predetermined moving pitch Mp in an array direction for each ultrasonic transmission, and performs the ultrasonic transmission from all the transducers 101a present in the probe 101. At that time, the transmitter 1031 cyclically changes a transmission condition for each ultrasonic transmission. The transmission condition is a condition defined by parameters such as a frequency of the ultrasonic wave to be transmitted, a transmission direction of the ultrasonic wave, a depth of a focal point, and a wave number of the ultrasonic wave, for example, and at least one of the above-described parameters is different among a plurality of transmission conditions. The transmitter 1031 uses four types of the transmission conditions of first to fourth transmission conditions, for example, and transmits under the second transmission condition while moving the transmission opening Tx in the array direction by the moving pitch Mp after transmitting under the first transmission condition, further transmits under the third transmission condition while moving the transmission opening Tx in the array direction by the moving pitch Mp, and transmits under the fourth transmission condition while moving the transmission opening Tx in the array direction by the moving pitch Mp. Then, if there is the transducers 101a present in the probe 101 from which the ultrasonic transmission is not performed, the transmission under the first transmission condition is performed while moving the transmission opening Tx by the moving pitch Mp in the array direction. Thereafter, the transmission is performed while moving the transmission opening Tx by the moving pitch Mp in the array direction while changing the transmission condition in order of the first, second, third, fourth, first, second, third and so on until the transmission opening Tx reaches an end of the array of the transducers 101a. The moving pitch Mp is preferably equal to or larger than a value obtained by dividing a width in the array direction of an acoustic line target area Bx to be described later by the number of transmission conditions and is equal to a width in the array direction of the transducer in the embodiment.

Information indicating a position of the transducer included in the transmission opening Tx is output to the data storage unit 109 via the controller 110. For example, when the total number of the transducers 101a present in the probe 101 is 192, 20 to 100 may be selected, for example, as the number of transducer arrays forming the transmission opening Tx, and it is possible to configure to move by the moving pitch Mp for each ultrasonic transmission. Hereinafter, a series of ultrasonic transmissions until the transmission opening Tx moves from one end to the other end of the transducer array 101a by the transmitter 1031 is collectively referred to as a “transmission/reception sequence”, and each ultrasonic transmission forming the transmission/reception sequence is referred to as a “transmission/reception event”.

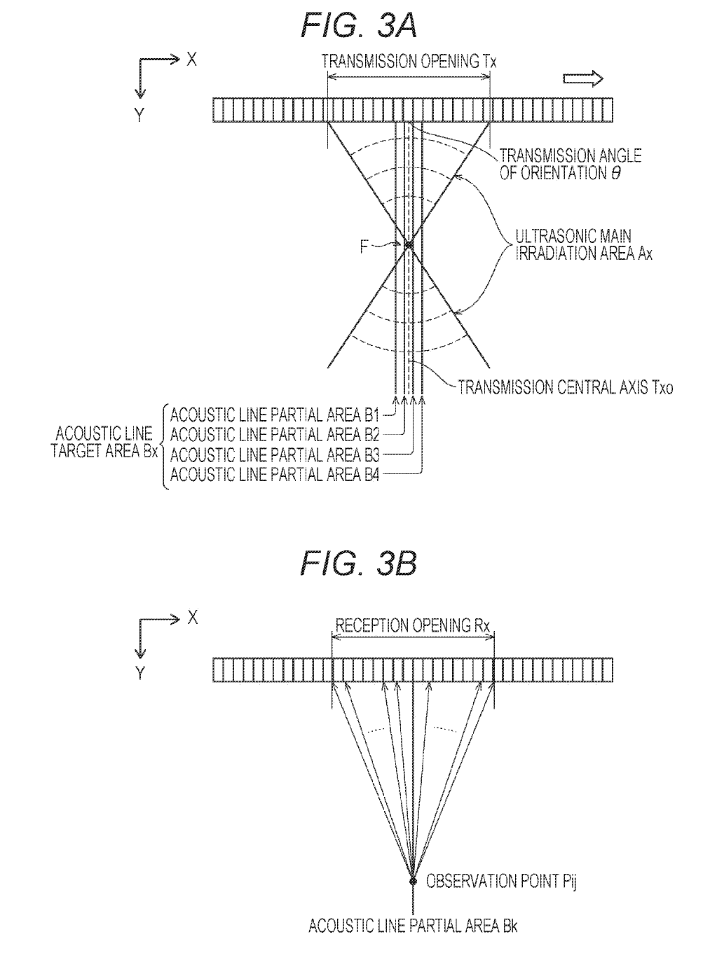

FIG. 3A is a schematic diagram illustrating a propagation path of an ultrasonic transmission wave by the transmission beam former 103. In a certain transmission/reception event, the array of the transducers 101a (transmission transducer array) arranged in an array contributing to the ultrasonic transmission is illustrated as the transmission opening Tx. Also, an array length of the transmission opening Tx is referred to as a transmission opening length. Also, the transmission direction of the ultrasonic wave is indicated by an angle θ between a straight line connecting the center of the transmission opening Tx and the focal point F and a normal direction of the transducer array 101a at the center of the transmission opening Tx. Note that the sign θ indicates inclination of the transmission direction in a positive direction or a negative direction of the transducer array direction (x direction), and a case of advancing in a positive x direction as it gets deeper (advancing toward lower right in the drawing) is set to positive and a case of advancing in a negative x direction as it gets deeper (advancing toward lower left in the drawing) is set to negative.

The transmission beam former 103 controls the transmission timing of each transducer such that the transmission timing of the transducer located closer to the center of the transmission opening Tx is more delayed. As a result, the ultrasonic transmission waves transmitted from the transducer array in the transmission opening Tx are such that a wave front is focused at a certain point, that is, the transmission focal point (F) at a certain depth (focal depth) of the subject. The depth (focal depth) of the transmission focal point F (hereinafter referred to as “transmission focal depth”) may be arbitrarily set. The wave front focused at the transmission focal point F diffuses again and the ultrasonic transmission waves propagate in an hourglass-shaped space separated by two intersecting straight lines with the transmission opening Tx as a bottom and the transmission focal point F as a node. That is, the ultrasonic waves emitted from the transmission opening Tx gradually reduce the width (horizontal axis direction in the drawing) thereof on the space, minimize the width at the transmission focal point F, and diffuse while widening the width again to propagate as they advance to a deeper portion (upper portion in the drawing). In other words, the hourglass-shaped area has a larger width as it is deeper than the focal depth. This hourglass-shaped area is an ultrasonic main irradiation area Ax.

2. Configuration of Reception Beam Former 104

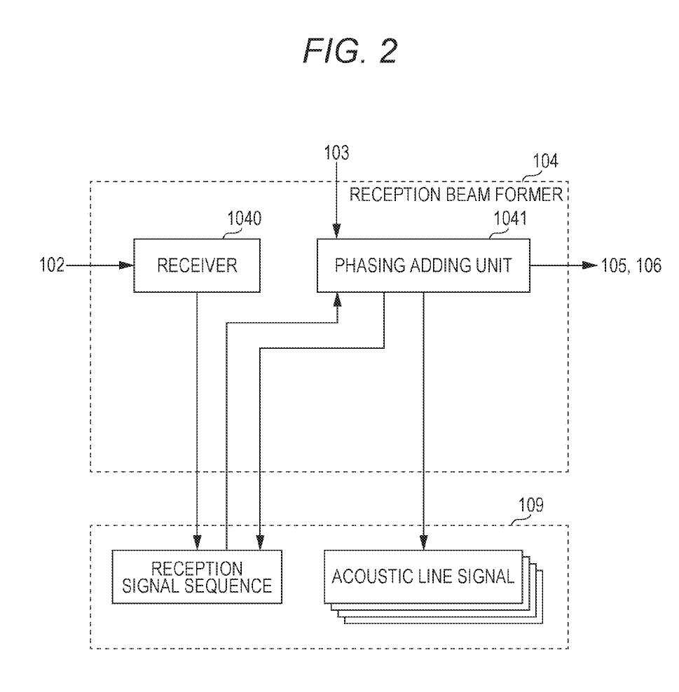

On the basis of the reflected waves of the ultrasonic waves received by the probe 101, the reception beam former 104 generates a subframe acoustic line signal from the electric signals obtained by a plurality of transducers 101a. Note that the “acoustic line signal” is a signal obtained after a phasing adding process is performed on a certain observation point. The phasing adding process is to be described later. FIG. 2 is a functional block diagram illustrating a configuration of the reception beam former 104. As illustrated in FIG. 2, the reception beam former 104 is provided with a receiver 1040 and a phasing adding unit 1041.

Hereinafter, a configuration of each unit forming the reception beam former 104 is described.

(1) Receiver 1040

The receiver 1040 is a circuit connected to the probe 101 via the multiplexer unit 102 which generates a reception signal (RF signal) obtained by amplifying the electric signals obtained by reception of the ultrasonic reflected waves by the probe 101 in synchronization with the transmission/reception event and then performing AD conversion. The reception signals are generated in chronological order in the order of transmission/reception events to be output to the data storage unit 109, and the reception signals are stored in the data storage unit 109.

Herein, the reception signal (RF signal) is a digital signal obtained by amplifying the electric signal converted from the reflected ultrasonic wave received by each transducer and performing the A/D conversion, and forms a sequence of signals continuous in the transmission direction (depth direction of the subject) of the ultrasonic wave received by each transducer.

In the transmission/reception event, as described above, the transmitter 1031 allows each of a plurality of transducers included in the transmission opening Tx out of a plurality of transducers 101a present in the probe 101 to transmit the ultrasonic beam. In contrast, the receiver 1040 generates a sequence of reception wave signals for each transducer on the basis of the reflected ultrasonic wave obtained by each of the transducers corresponding to a part or all of a plurality of transducers 101a present in the probe 101 in synchronization with the transmission/reception event. Herein, the transducer which receives the reflected ultrasonic wave is referred to as a “reception transducer”. It is preferable that the number of reception transducers is larger than the number of transducers included in the transmission opening Tx. In addition, the number of reception transducers may be the total number of transducers 101a present in the probe 101.

The receiver 1040 generates the sequence of reception signals for each reception transducer in synchronization with the transmission/reception event, and the generated reception signal is stored in the data storage unit 109.

(2) Phasing Adding Unit 1041

The phasing adding unit 1041 generates a subframe acoustic line signal in the subject in synchronization with the transmission/reception event. Specifically, as illustrated in FIG. 3A, an acoustic line target area Bx including linear acoustic line partial areas B1 to B4 is set depending on the positions of the transmission focal point F and the transmission opening Tx. The number of acoustic line partial areas Bk (k is an integer) is preferably the number of transmission conditions or an integer multiple thereof. In this embodiment, the acoustic line partial areas B1 to B4 which are areas extended in a direction of propagation of the ultrasonic wave from four consecutive transducers which are two transducers the closest to the center of the transmission opening Tx and two transducers adjacent to the transducers are set. That is, a width in the array direction of the acoustic line target area Bx is four times the width of the transducer.

Next, for each of a plurality of observation points Pij present on the acoustic line target area Bx, the phasing adding unit 1041 performs phasing addition on the reception signal sequence received by each reception transducer from the observation point. Specifically, as illustrated in FIG. 3B, a reception opening Rx is set for the observation point Pij. The reception opening Rx is selected such that the center of the reception opening Rx is a transducer Xk spatially the closest to the observation point Pij. Alternatively, the reception opening Rx may be set such that the center of the reception opening Rx and the center of the transmission opening Tx coincide with each other. Then, the phasing adding unit 1041 calculates the delay time for each transducer Rk on the basis of a transmission time until the ultrasonic wave reaches the observation point Pij from the transmission opening Tx and a reception time for each transducer Rk until the reflected ultrasonic wave reaches each transducer Rk included in the reception opening Rx from the observation point Pij. Then, the acoustic line signal corresponding to Pij is generated by identifying signals corresponding to the observation point Pij from the reception signal sequence by using the delay time and adding them.

The receiver 1040 generates the acoustic line signals corresponding to the acoustic line target area Bx in synchronization with the transmission/reception event, and the generated acoustic line signals are stored in the data storage unit 109.

3. Configuration of CFM Processor 105

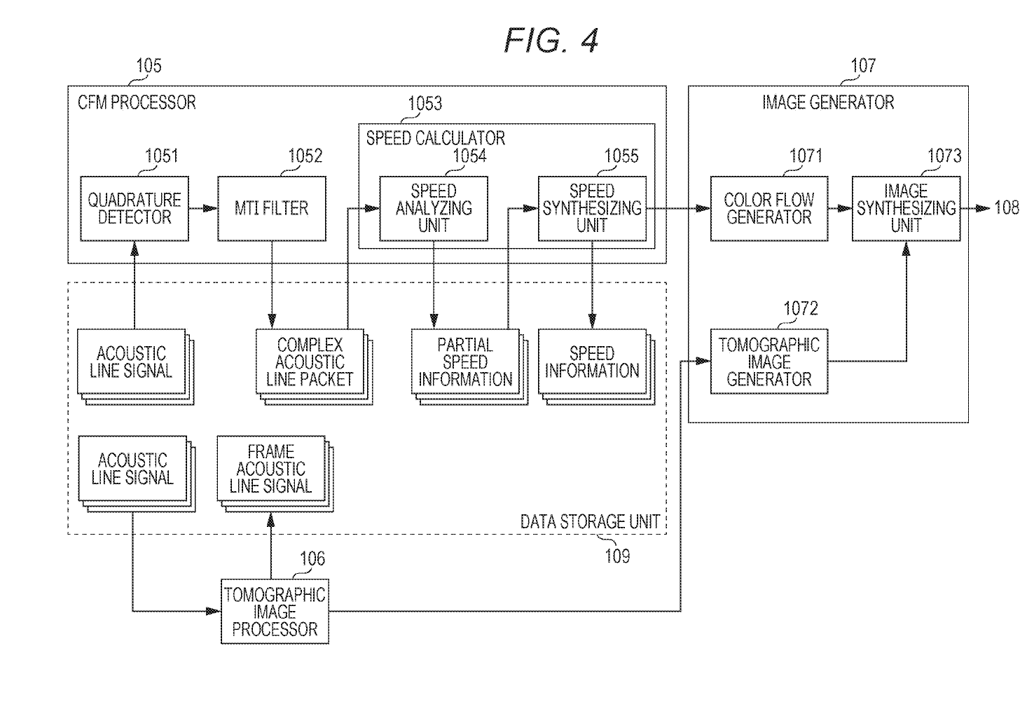

The CFM processor 105 performs frequency analysis on the basis of a plurality of acoustic line signals obtained in each of a plurality of transmission/reception events to generate a CFM signal. Note that the “CFM signal” is a signal indicating speed information for a certain observation point. The speed information is to be described later. FIG. 4 is a functional block diagram illustrating configurations of the CFM processor 105, the tomographic image processor 106, and the image generator 107. As illustrated in FIG. 4, the CFM processor 105 is provided with a quadrature detector 1051, a filter unit 1052, and a speed calculator 1053.

Hereinafter, a configuration of each unit forming the CFM processor 105 is described.

(1) Quadrature Detector 1051

The quadrature detector 1051 is a circuit which performs quadrature detection on each of the acoustic line signals generated in synchronization with the transmission/reception event and generates a complex acoustic line signal indicating a phase of the reception signal at each observation point. Specifically, the following process is performed. First, a first reference signal the frequency of which is the same as that of the transmission ultrasonic wave and a second reference signal having the same frequency and amplitude as those of the first reference signal in which only a phase is different by 90 degrees are generated. Next, the acoustic line signal and the first reference signal are integrated, and a high-frequency component having a frequency approximately twice the frequency of the first reference signal is removed by LPF to obtain a first component. Similarly, the acoustic line signal and the second reference signal are integrated, and a high-frequency component having a frequency approximately twice the frequency of the second reference signal is removed by LPF to obtain a second component. Finally, the complex acoustic line signal is generated with the first component as a real part (I component; in phase) and the second component as an imaginary part (Q component: quadrature phase).

(2) Filter Unit 1052

The filter unit 1052 is a filter circuit which removes clutter from the complex acoustic line signal. The clutter is a component that is not a target to be imaged among tissue movement, specifically, information indicating the movement of tissue such as a blood vessel wall, a muscle, and an organ. The clutter is larger in power than a signal indicating a blood flow, but the movement of tissue is slower than the blood flow, so that the frequency thereof is lower than that of the signal indicating the blood flow. Therefore, it is possible to selectively remove only the clutter. A known so-called “wall filter” and “moving target indicator (MTI) filter” may be applied to the filter unit 1052.

The filter unit 1052 stores the filtered complex acoustic line signals as a complex acoustic line packet for each transmission condition in the data storage unit 109.

(3) Speed Calculator 1053

The speed calculator 1053 is a circuit which estimates movement in the subject, specifically the blood flow corresponding to each observation point from the complex acoustic line signal after the filter process. For each observation point, the speed calculator 1053 estimates the phase from the complex acoustic line signals corresponding to a plurality of transmission/reception events related to a plurality of transmission/reception sequences, and calculates a change speed of the phase. The speed calculator 1053 is provided with a speed analyzing unit 1054 and a speed synthesizing unit 1055.

The speed analyzing unit 1054 obtains the complex acoustic line signals for each transmission condition and performs speed analysis. Specifically, the speed analyzing unit 1054 first reads the complex acoustic line signals obtained by the transmission/reception events under the first transmission condition from the complex acoustic line signals related to a plurality of transmission/reception sequences as a first complex acoustic line packet and calculates a speed vi for each observation point Pij by using the same as ensemble data. In this embodiment, the complex acoustic line signals related to the transmission/reception events under the first transmission condition out of the complex acoustic line signals related to the latest transmission/reception sequence, the precedent transmission/reception sequence, two transmission/reception sequences before, and three transmission/reception sequences before as the first complex acoustic line packet. As a method of calculating the speed, it is possible to specify the phase of the complex acoustic line signal and estimate a change speed of the phase, and it is possible to estimate the change speed of the phase by performing a correlation process between the complex acoustic line signals. The speed analyzing unit 1054 stores the calculated speed vi in the data storage unit 109 as partial speed information.

Next, the speed analyzing unit 1054 reads the complex acoustic line signals obtained by the transmission/reception events under the second transmission condition from the complex acoustic line signals related to a plurality of transmission/reception sequences as a second complex acoustic line packet and calculates a speed v₂ for each observation point Pij by using the same as ensemble data. The speed analyzing unit 1054 stores the calculated speed v₂ in the data storage unit 109 as partial speed information.

Similarly, the speed analyzing unit 1054 reads the complex acoustic line signals obtained by the transmission/reception events under the third transmission condition from the complex acoustic line signals related to a plurality of transmission/reception sequences as a third complex acoustic line packet and calculates a speed v₃ for each observation point Pij by using the same as ensemble data. The speed analyzing unit 1054 stores the calculated speed v₃ in the data storage unit 109 as partial speed information.

Furthermore, the speed analyzing unit 1054 reads the complex acoustic line signals obtained by the transmission/reception events under the fourth transmission condition from the complex acoustic line signals related to a plurality of transmission/reception sequences as a fourth complex acoustic line packet and calculates a speed v₄ for each observation point Pij by using the same as ensemble data. The speed analyzing unit 1054 stores the calculated speed v₄ in the data storage unit 109 as partial speed information.

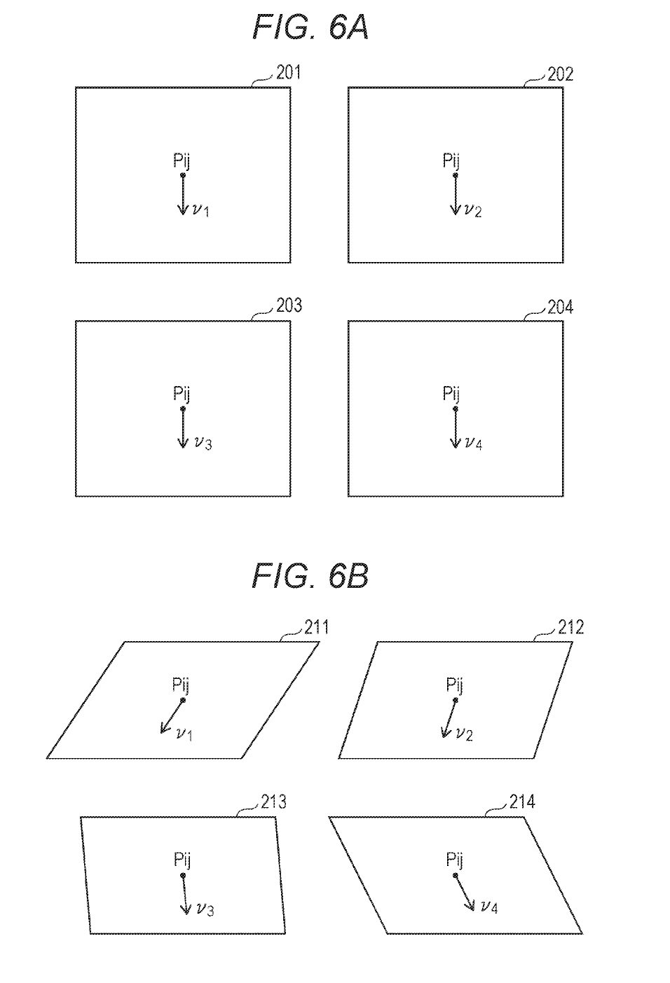

The speed synthesizing unit 1055 obtains a plurality of pieces of partial speed information created on the basis of the same transmission/reception sequence and synthesizes the speeds with the position of the observation point Pij as an index. Specifically, as illustrated in FIG. 6A, partial speed information 201 corresponding to the first transmission condition, partial speed information 202 corresponding to the second transmission condition, partial speed information 203 corresponding to the third transmission condition, and partial speed information 204 corresponding to the fourth transmission condition are read from the data storage unit 109. Then, for the observation point Pij corresponding to the same place in the subject, the speed value v is calculated on the basis of the following expression.

v=αv₁+βv₂+γv₃+δv₄,

wherein α+β+γ+δ=1. Each of weighting coefficients α, β, γ, and δ may be constant irrespective of the position of the observation point Pij or may change depending on the position of the observation point Pij. As an example of the weighting coefficients, for example, the weighting coefficient is made larger for the transmission condition in which an absolute value of the transmission direction θ is smaller. By doing so, it is possible to obtain a benefit of space compounding while improving sensitivity to the movement in the depth direction (Y direction) in which detection is desired to be performed.

Also, for example, the weighting is made larger as the position of the observation point Pij is shallower in the transmission condition with a high ultrasonic frequency, and the weighting is made larger as the position of the observation point Pij is deeper in the transmission condition with a low ultrasonic frequency. By doing so, it is possible to obtain the movement in a shallow portion with a high degree of accuracy by using a high-frequency ultrasonic wave which attenuates significantly while having high resolution, and detect the movement by using a low-frequency ultrasonic wave in a deep portion in which the high-frequency ultrasonic wave does not sufficiently propagate. Also, for example, the weighting is made larger as the depth of the observation point Pij is closer to the transmission focal depth. By doing so, it is also possible to calculate the speed on the basis of the acoustic line signal having a high S/N ratio near the transmission focal point.

The speed synthesizing unit 1055 outputs the speed value v for each observation point Pij as the speed information to the image generator 107 and the data storage unit 109. Note that the speed synthesizing unit 1055 may further calculate a variance value of the speed and power on the basis of the synthesized speed value v and similarly output the same to the image generator 107 and the data storage unit 109.

4. Configuration of Tomographic Image Processor 106

The tomographic image processor 106 synthesizes the acoustic line signals obtained by a plurality of transmission/reception events, and generates a frame acoustic line signal which is a synthesized acoustic line signal of one frame. The tomographic image processor 106 outputs the frame acoustic line signal to the image generator 107 and the data storage unit 109.

5. Configuration of Image Generator 107

The image generator 107 is a circuit for generating the color Doppler image by converting the frame acoustic line signal generated by the tomographic image processor 106 into the B-mode tomographic image and performing color tone conversion on the frame CFM signal generated by the CFM processor 105 to superimpose on the same. As illustrated in FIG. 4, the image generator 107 is provided with a color flow generator 1071, a tomographic image generator 1072, and an image synthesizing unit 1073.

(1) Color Flow Generator 1071

The color flow generator 1071 is a circuit which performs the color tone conversion for generating the color Doppler image from the frame CFM signal. Specifically, a coordinate system of the frame CFM signal is first converted into an orthogonal coordinate system. Next, an average speed at each observation point is converted into color information to generate color flow information. At that time, it is converted such that, for example, (1) a direction toward the probe is in red and a direction away from the probe is in blue and (2) saturation is higher as an absolute value of the speed is larger and saturation is lower as the absolute value is smaller. More specifically, regarding a speed component toward the probe, the absolute value of the speed is converted into a red luminance value, and regarding a speed component away from the probe, the absolute value of the speed is converted into a blue luminance value.

Note that the color flow generator 1071 may further receive a signal indicating speed variance from the CFM processor 105 and convert the variance value into a green luminance value. By doing so, it is possible to indicate a position where turbulence occurs.

The color flow generator 1071 outputs the generated color flow information to the image synthesizing unit 1073.

(2) Tomographic Image Generator 1072

The tomographic image generator 1072 is a circuit which generates the B-mode tomographic image from the frame acoustic line signal. Specifically, the coordinate system of the frame acoustic line signal is first converted into the orthogonal coordinate system. Next, the value of the acoustic line signal at each observation point is converted to luminance to generate the B-mode tomographic image. Specifically, the tomographic image generator 1072 performs envelope detection on the value of the acoustic line signal and performs logarithmic compression to convert the same into luminance The tomographic image generator 1072 outputs the generated B-mode tomographic image to the image synthesizing unit 1073.

(2) Image Synthesizing Unit 1073

The image synthesizing unit 1073 is a circuit which generates the color Doppler image by superimposing the color flow information generated by the color flow generator 1071 on the B-mode tomographic image generated by the tomographic image generator 1072, and outputs the same to the display unit 108. As a result, the color

Doppler image obtained by adding a direction and speed (absolute value of speed) of the blood flow on the B-mode tomographic image is displayed on the display unit 108.

### Transmission/Reception Event in Transmission Beam Former 103 in Detail

Hereinafter, order of execution and the transmission conditions of the transmission/reception events are described in detail.

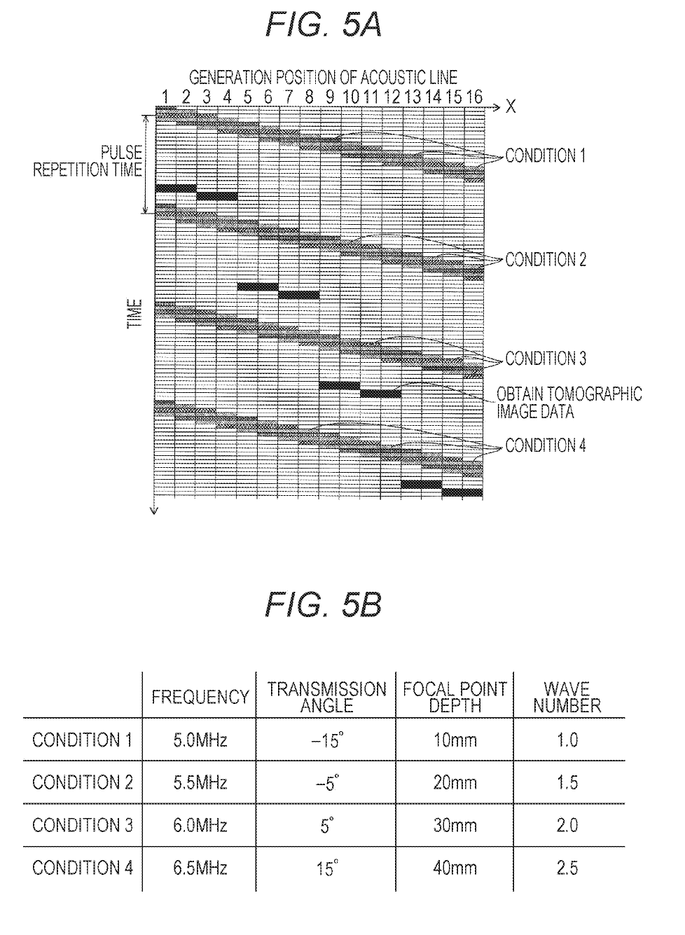

FIG. 5A is a time chart illustrating a relationship between an execution time of the transmission/reception event and a transmission/reception area of the ultrasonic waves. FIG. 5B is a view illustrating an example of the transmission conditions. Note that, in FIG. 5B, four parameters of the ultrasonic frequency, the transmission direction, the focal depth, and the wave number are illustrated as the parameters of the transmission conditions, but it is only required that one of the parameters is different between arbitrary two transmission conditions, and it is not required that all the four parameters be different.

The position of the transmission focal point F in an element array direction (x direction) and a generation position of the acoustic line are plotted along the horizontal axis (X axis) in FIG. 5A. Note that, in FIG. 5A, the number of transducers is 16, the number of types of transmission conditions is four, and the number of acoustic lines generated for each transmission/reception event is four, but there is no limitation.

A first transmission/reception sequence is first described. As the first transmission/reception event, the transmission beam former 103 performs the ultrasonic transmission under the first transmission condition while setting a position on a left side of the first transducer by 1.5 transducers as the center of the transmission opening Tx. In synchronization with this, the reception beam former 104 generates the acoustic line for a linear area passing through the first transducer on the basis of the reflected ultrasonic wave. Next, at a time when a first time elapses from the ultrasonic transmission in the first transmission/reception event, the transmission beam former 103 performs the ultrasonic transmission under the second transmission condition while setting a position on a left side of the first transducer by 0.5 transducers as the center of the transmission opening Tx. In synchronism with this, the reception beam former 104 generates the acoustic lines for the linear areas passing through the first and second transducers, respectively, on the basis of the reflected ultrasonic waves. Next, at a time when the first time elapses from the ultrasonic transmission in the second transmission/reception event, the transmission beam former 103 performs the ultrasonic transmission under the third transmission condition while setting an intermediate position between the first transducer and the second transducer as the center of the transmission opening Tx. In synchronism with this, the reception beam former 104 generates the acoustic lines for the linear areas passing through the first, second, and third transducers, respectively, on the basis of the reflected ultrasonic waves. At a time when the first time elapses from the ultrasonic transmission in the third transmission/reception event, the transmission beam former 103 performs the ultrasonic transmission under the fourth transmission condition while setting an intermediate position between the second transducer and the third transducer as the center of the transmission opening Tx. In synchronism with this, the reception beam former 104 generates acoustic lines for the linear areas passing through the first, second, third, and fourth transducers, respectively, on the basis of the reflected ultrasonic waves. At time when the first time elapses from the ultrasonic transmission in the fourth transmission/reception event, the transmission beam former 103 performs the ultrasonic transmission under the first transmission condition while setting an intermediate position between the third transducer and the fourth transducer as the center of the transmission opening Tx. In synchronism with this, the reception beam former 104 generates the acoustic lines for the linear areas passing through the second, third, fourth, and fifth transducers, respectively, on the basis of the reflected ultrasonic waves. Hereinafter, similarly, the transmission/reception event is performed such that four conditions of (1) an interval of the ultrasonic transmissions is a prescribed first time, (2) the transmission condition is cyclically changed for each ultrasonic transmission in determined order such as first, second, third, fourth, first, second and so on, (3) the position of the transmission opening Tx moves in the x direction by a fixed pitch Mp (in this embodiment, by one transducer) each time the ultrasonic transmission is performed, and (4) the acoustic line target area Bx moves in the x direction in synchronization with the position of the center of the transmission opening Tx are satisfied. Note that, in a case where the transmission direction is used as the parameter in the transmission condition, as for the above-described (4), a portion with depth of 0 in the acoustic line target area Bx moves in the x direction in synchronization with the position of the center of the transmission opening Tx, and a shape of the acoustic line target area Bx changes in synchronous with the transmission direction.

When a 16th transmission/reception event using the fourth transmission condition is performed, all of the acoustic line corresponding to the first transmission condition, the acoustic line corresponding to the second transmission condition, the acoustic line corresponding to the third transmission condition, and the acoustic line corresponding to the fourth transmission condition are generated in all of the linear areas passing through one transducer. As a result, the first transmission/reception sequence ends.

Then, at a time when a second time (pulse repetition time) elapses after the first transmission/reception sequence is started, a second transmission/reception sequence is started.

Note that, after the first transmission/reception sequence ends until the second transmission/reception sequence starts, a partial transmission/reception sequence which is one-quarter of the transmission/reception sequence for generating the tomographic image is performed, and the acoustic lines are generated for the linear areas passing through the first, second, third, and fourth transducers, respectively. Similarly, after the second transmission/reception sequence ends until the third transmission/reception sequence starts, the partial transmission/reception sequence which is continuous one-quarter of the transmission/reception sequence for generating the tomographic image is performed, and the acoustic lines are generated for the linear areas passing through the fifth, sixth, seventh, and eighth transducers, respectively. After the third transmission/reception sequence ends until the fourth transmission/reception sequence starts, the partial transmission/reception sequence which is continuous one-quarter of the transmission/reception sequence for generating the tomographic image is performed, and the acoustic lines are generated for the linear areas passing through the ninth, tenth, eleventh, and twelfth transducers, respectively. After the fourth transmission/reception sequence ends until the fifth transmission/reception sequence starts, the partial transmission/reception sequence which is continuous one-quarter of the transmission/reception sequence for generating the tomographic image is performed, and the acoustic lines are generated for the linear areas passing through the 13th, 14th, 15th, and 16th transducers, respectively. Note that, in the partial transmission/reception sequence, only the movement in the x direction of the center position of the transmission opening Tx and the movement of the acoustic line target area Bx synchronized therewith are performed for each transmission/reception event, and the transmission condition is constant.

By the above-described operation, the tomographic image of one frame may be generated by the four partial transmission/reception sequences. In contrast, as for the color flow information, the tomographic image of one frame may be generated by one transmission/reception sequence. This is because the color flow information may be generated on the basis of the first, second, third, and fourth transmission/reception sequences when the fourth transmission/reception sequence ends, and the color flow information may be generated on the basis of the second, third, fourth, and fifth transmission/reception sequences at the time when the fifth transmission/reception sequence ends.

Also, in each transmission/reception sequence, spatial density in the x direction at the observation point in the acoustic line target area Bx is the same as for the acoustic lines corresponding to the first transmission condition, the acoustic lines corresponding to the second transmission condition, the acoustic lines corresponding to the third transmission condition, and the acoustic lines corresponding to the fourth transmission condition, and this also coincides with a case where all the transmissions are performed under the same transmission condition. In contrast, a time required for the transmission/reception sequence increases only by a time required for (the number of transmission conditions −1)×one transmission/reception events. That is, it is possible to obtain the acoustic line signals corresponding to various transmission conditions without significantly increasing the time required for the transmission/reception sequence and without lowering the spatial density in the x direction at the observation point in the acoustic line target area Bx.

### Operation

Operation of the ultrasonic diagnostic device 100 having the above-described configuration is described.

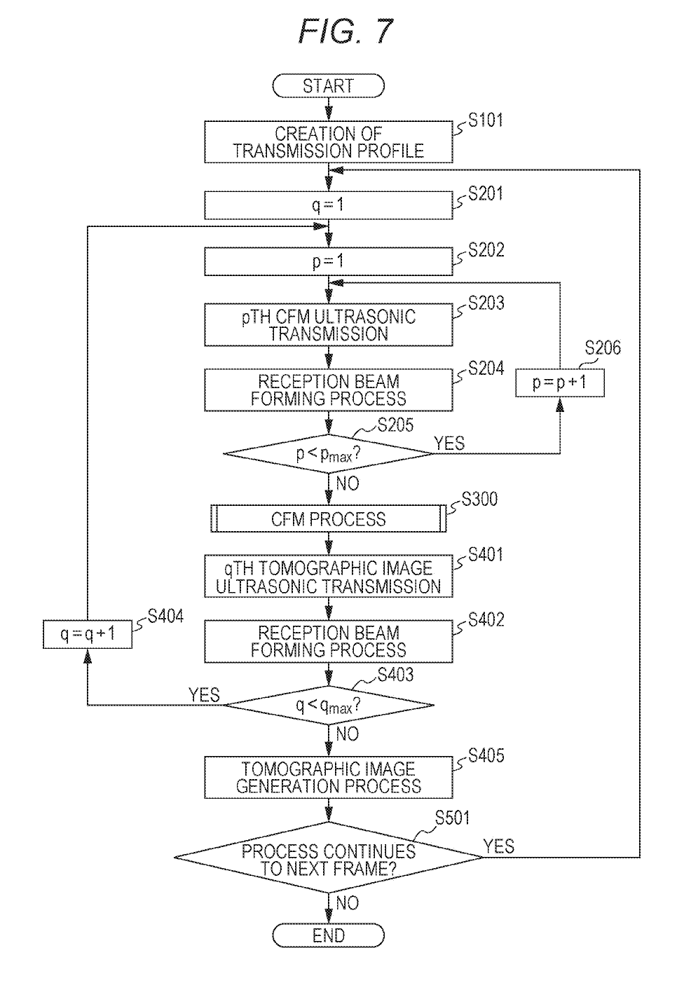

FIG. 7 is a flowchart illustrating the operation of the ultrasonic diagnostic device 100.

First, at step S101, a transmission profile is created. The transmission profile is information which defines two or more transmission conditions, the moving pitch Mp and the number of acoustic lines of the transmission opening Tx for each transmission/reception event, and the number of partial transmission/reception sequences.

Next, a counter q of the transmission/reception sequence is initialized to one (step S201), and the first transmission/reception sequence is started.

Next, a counter p of the transmission/reception event is initialized to one (step S202), and the ultrasonic transmission in a pth transmission/reception event (step S203) and reception beam forming synchronized therewith (step S204) are executed.

Next, at step S205, it is determined whether the counter p of the transmission/reception event reaches the number of transmission/reception events during the transmission/reception sequence pₘₐₓ. When the counter p of the transmission/reception event is smaller than pₘₐₓ, p is incremented (step S206) and the next transmission/reception event is executed. In the next transmission/reception event, the transmission condition, the position of the transmission opening Tx, and the generation position of the acoustic line are changed. As a result, all the transmission/reception events included in one transmission/reception sequence are executed in order. When all the transmission/reception events included in one transmission/reception sequence are executed, the counter p of the transmission/reception event coincides with pₘₐₓ, so that the procedure shifts to step S300.

Next, at step S300, the CFM process is executed.

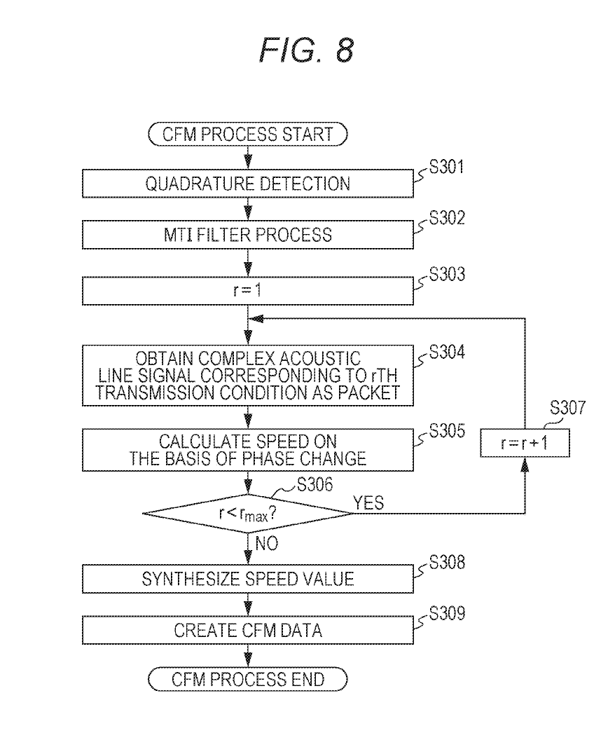

Herein, the CFM process at step S300 is described. FIG. 8 is a flowchart illustrating the CFM process in the CFM processor 105.

First, at step S301, the quadrature detector 1051 performs quadrature detection on each of the acoustic line signals to generate the complex acoustic line signal.

Next, at step S202, the filter unit 1052 removes or reduces the clutter component from the complex acoustic line signal.

Next, at step S303, a counter r of the transmission condition is initialized to one. At step S304, the speed analyzing unit 1054 obtains the complex acoustic line signals corresponding to an rth transmission condition over a plurality of transmission/reception sequences, and obtains the same as the packet. Then, at step S305, the speed analyzing unit 1054 performs a correlation process between a plurality of complex acoustic line signals related to the same observation point P included in the packet, estimates the change speed of the phase to calculate the speed, and outputs the same as the partial speed information to the data storage unit 109.

Next, at step S306, it is determined whether the counter r of the transmission condition reaches the number of the transmission conditions r_{max.} When the counter r of the transmission condition is smaller than rₘₐₓ, r is incremented (step S307), and the speed is calculated for the next transmission condition to be output to the data storage unit 109 as the partial speed information. As a result, the speed for each observation point P is calculated for each transmission condition. When the speed is calculated for all the transmission conditions, the counter r of the transmission condition coincides with rₘₐₓ, so that the procedure shifts to step S309.

Next, at step S309, the speed synthesizing unit 1055 synthesizes a plurality of pieces of partial speed information with the position of the observation point as an index to generate the speed information. The image generator 107 updates and displays the color flow information on the basis of the generated speed information.

Returning to FIG. 7, the description is continued. After execution of the transmission/reception sequence and the subsequent CFM process, the ultrasonic diagnostic device 100 performs a qth partial transmission/reception sequence related to the tomographic image (step S401), and performs the reception beam forming in synchronization therewith (step S402).

Next, at step S403, it is determined whether the counter q of the transmission/reception sequence reaches the number of partial transmission/reception sequence qₘₐₓ. When the counter q of the transmission/reception event is smaller than qₘₐₓ, q is incremented (step S404) and the next transmission/reception sequence is executed. In the next transmission/reception sequence, the position of the transmission opening Tx and the generation position of the acoustic line in the partial transmission/reception sequence are changed. When all the partial transmission/reception sequences are executed, the counter q of the transmission/reception sequence coincides with qₘₐₓ, so that the procedure shifts to step S405.

Next, at step S405, a tomographic image generating process is performed. The tomographic image generator 1072 generates the frame acoustic line signal from the acoustic line signals related to all the partial transmission/reception sequences. The image generator 107 updates and displays the tomographic image on the basis of the generated frame acoustic line signal.

Finally, the ultrasonic diagnostic device 100 determines whether to continue the process to a next frame (step S501), and in a case of continuing, the procedure returns to step S201.

### SUMMARY

As described above, according to the ultrasonic diagnostic device 100 according to this embodiment, the speed analysis is performed on the basis of a plurality of acoustic line signals regarding the observation point P at the same position generated by the transmission/reception event under the same transmission condition, and a plurality of analysis results obtained under a plurality of transmission conditions are synthesized to obtain the speed information. As a result, it is possible to obtain the speed analysis result on the basis of an appropriate transmission condition for each observation point, and improve analysis accuracy of an entire target area.

Also, in the ultrasonic diagnostic device 100, by moving the transmission opening Tx and the acoustic line target area Bx while sequentially changing the transmission condition in one transmission/reception sequence, (1) the time interval of the transmission/reception events performed under the same transmission condition is constant, and (2) the density in the element array direction (x direction) of the acoustic lines is constant between the transmission conditions. Therefore, the pulse repetition time becomes constant for any observation point P, and there is no variation in sensing accuracy for low speed movement within the target area. In addition, since the CFM process using a plurality of transmission conditions is performed without causing a decrease in density in the element array direction (x direction) of the acoustic lines and hardly elongating the time required for one transmission and reception sequence, it is possible to improve the analysis accuracy of the entire target area without lowering spatial resolution and a frame rate.

### First Variation

In the ultrasonic diagnostic device 100 according to the first embodiment, as illustrated in the time charts of FIGS. 5A and 5B, it is configured such that all the transmission condition, the center position of the transmission opening Tx, and the acoustic line target area Bx are changed for each transmission/reception event. However, the center position of the transmission opening Tx and the acoustic line target area Bx may be changed as appropriate as follows.

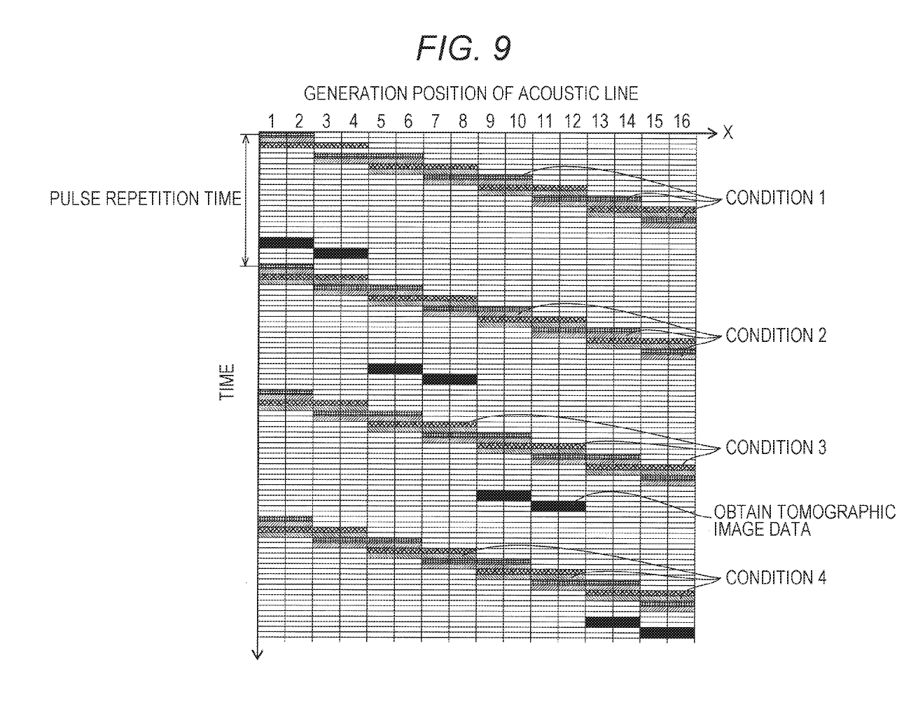

A time chart of FIG. 9 is a first example illustrating a relationship between an execution time of the transmission/reception event and a transmission/reception area of ultrasonic waves. In the first embodiment, it is configured such that the center position of the transmission opening Tx is changed for each transmission/reception event, and the acoustic line target area Bx is changed in synchronization with this; however, in the first example, the center position of the transmission opening Tx and the acoustic line target area Bx are changed each time the transmission/reception event is performed twice. That is, between the first transmission/reception event and the second transmission/reception event, only the transmission conditions are different, and the center position of the transmission opening Tx and the acoustic line target area Bx are the same. Also, similarly, between a third transmission/reception event and a fourth transmission/reception event, only the transmission conditions are different, and the center position of the transmission opening Tx and the acoustic line target area Bx are the same. On the other hand, between the second transmission/reception event and the third transmission/reception event, the center position of the transmission opening Tx moves by two transducers. That is, a moving pitch Mp is twice a value obtained by dividing a width in an array direction of the acoustic line target area Bx to be described later by the number of transmission conditions or larger.

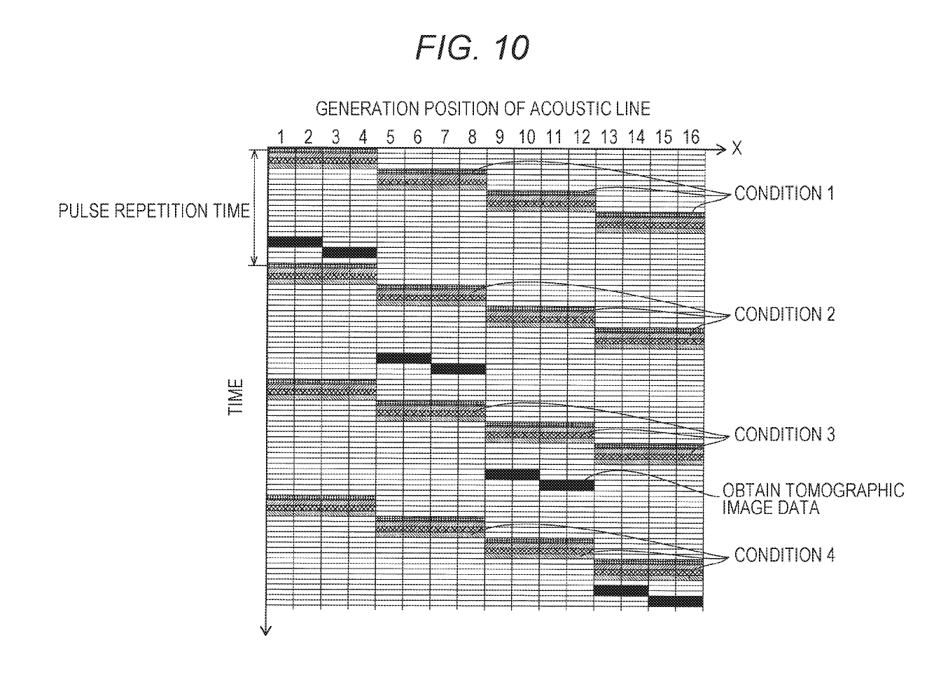

A time chart of FIG. 10 is a second example illustrating a relationship between the execution time of the transmission/reception event and the transmission/reception area of the ultrasonic waves. In the second example, the center position of the transmission opening Tx and the acoustic line target area Bx are changed each time the transmission/reception event is performed four times. That is, among the first to fourth transmission/reception events, only the transmission conditions are different, and the center position of the transmission opening Tx and the acoustic line target area Bx are the same. Also, similarly, among fifth to eighth transmission/reception events, only the transmission conditions are different, and the center position of the transmission opening Tx and the acoustic line target area Bx are the same. On the other hand, between the fourth transmission/reception event and the fifth transmission/reception event, the center position of the transmission opening Tx moves by four transducers. That is, the moving pitch Mp is four times the value obtained by dividing the width in the array direction of the acoustic line target area Bx to be described later by the number of transmission conditions or larger.

Also in the operation as described above, (1) an interval of ultrasonic transmissions is a prescribed first time, and (2) the transmission condition is cyclically changed for each ultrasonic transmission in predetermined order such as first, second, third, fourth, first, second and so on are the same as those in the first embodiment. As a result, in linear areas passing through all the transducers, all the acoustic line corresponding to a first transmission condition, the acoustic line corresponding to a second transmission condition, the acoustic line corresponding to a third transmission condition, and the acoustic line corresponding to a fourth transmission condition are generated.

### Second Variation

In the ultrasonic diagnostic device 100 according to the first embodiment, the speed value v is calculated for all Pij in the subject on the basis of the following equation.

v=αv₁+βv₂+γv₃+δv₄

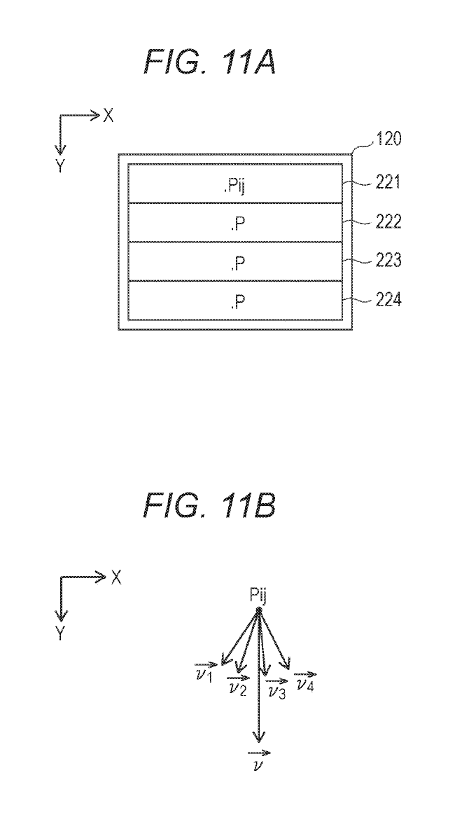

However, one or more of weighting coefficients α, β, γ, and δ may be zero. For example, in a case of using a transmission direction as a transmission condition, in a transmission/reception event in which a sign of a transmission direction θ is positive, an acoustic line target area Bx moves in a positive x direction as it is deeper (in the drawing, a lower side inclines to a right side), so that it is not possible to obtain an acoustic line signal in an area with a small x coordinate (an area on a lower left side in the drawing) in a deep area. In such a case, for example, the weighting coefficient related to the transmission condition with which a speed value is not calculated may be set to zero. Also, for example, in a case of using a depth of a transmission focal point as the transmission condition, as illustrated in FIG. 11A, a target area may be divided into four areas 221, 222, 223, and 224 according to the depth, and the area 221 may be such that α=1, β=0, γ=0, and δ=0, the area 222 may be such that α=0, β=1, γ=0, and δ=0, the area 223 may be such that α=0, β=0, γ=1, and δ=0, and the area 224 may be such that α=0, β=0, γ=0, and δ=1. By doing so, it is possible to optimize the transmission condition without speed synthesis calculation.

Also, although it is calculated while setting the speed value as a one-dimensional vector in the depth direction in the speed value synthesis in the ultrasonic diagnostic device 100 according to the first embodiment, it is also possible to perform two-dimensional vector calculation as illustrated in FIG. 11B. That is, in a case of using the transmission direction as the transmission condition, the speed value at the observation point Pij corresponding to a first transmission condition is assumed to be a vector v₁ having a direction in a propagation direction of the ultrasonic wave under the first transmission condition. Herein, the direction in the propagation direction of the ultrasonic wave is a direction parallel to a vector from the center of the transmission opening Tx to a transmission focal point F. Similarly, by setting a speed value corresponding to a second transmission condition as a vector v₂, setting a speed value corresponding to a third transmission condition as a vector v₃, and setting a speed value corresponding to a fourth transmission condition as a vector v₄, and a speed vector v is calculated by vector synthesis. By doing so, it is possible to calculate the speed value accurately also for the movement not parallel to the depth direction. Also, in a power Doppler mode indicating an energy value of the movement, it is possible to detect and display also energy caused by movement in a horizontal direction orthogonal to the depth direction, so that the existence of fine blood vessels may be rendered more accurately. Note that, in this vector synthesis also, weighting addition may be performed.

### Other Variations According to Embodiment

(1) In the embodiment and the variations, the number of transmission conditions is set to four, but the number of transmission conditions may be an arbitrary number not smaller than two. However, in a case where the number of transmission conditions is set to n (n is an integer not smaller than three), it is preferable that a transmission/reception sequences are in order of a transmission/reception event under a first transmission condition, the transmission/reception event under a second transmission condition, . . . , the transmission/reception event under an nth transmission condition, the transmission/reception event under the first transmission condition and so on. As a result, a pulse repetition time is the same for any observation point and it is possible to suppress variation in acoustic line density between the transmission conditions by simple operation of moving the center position of a transmission opening Tx and an acoustic line target area Bx by a fixed pitch Mp each time the transmission/reception event is performed m times (m is an integer not smaller than one). Also, by setting a width in an x direction of the acoustic line target area Bx to be Mp/m or larger, it is possible to suppress occurrence of an area having low acoustic line density in a packet.

Also, similarly, the number of partial transmission/reception sequences related to a tomographic image is not limited to four, and may be an arbitrary number.

(2) In the embodiment and the variations, the color flow generator 1071 converts the average speed at each observation point to the color information to generate the color Doppler image, but the present invention is not necessarily limited to this case. For example, it is also possible that the speed calculator 1053 calculates power from a power spectrum at each observation point to generate a frame power signal, and the color flow generator 1071 converts the power value into a yellow luminance value, thereby generating the power Doppler image.

(3) Note that, although the present invention is described on the basis of the above-described embodiment, the present invention is not limited to the above-described embodiment, and the following cases are also included in the present invention.

For example, the present invention may be a computer system provided with a microprocessor and a memory, the memory storing a computer program, and the microprocessor operating according to the computer program. For example, this may be a computer system including a computer program of a diagnostic method of the ultrasonic diagnostic device of the present invention which operates according to the program (or instructs each connected site to operate).

Even a case where an entire or a part of the above-described ultrasonic diagnostic device, or an entire or a part of a beam former is of a computer system including a microprocessor, a recording medium such as a ROM and a RAM, and a hard disk unit is also included in the present invention. In the above-described RAM or hard disk unit, a computer program for realizing the operation similar to that of each of the above-described devices is stored. The above-described microprocessor operates according to the above-described computer program, so that each device realizes its function.

In addition, a part of or all of the components forming each of the above-described devices may be of one system large scale integration (LSI). The system LSI is a super multifunctional LSI manufactured by integrating a plurality of components on one chip, and specifically is a computer system including a microprocessor, a ROM, a RAM and the like. They may be separately formed into one chip, or may be formed into one chip so as to include a part or all of them. Note that the LSI is sometimes referred to as an IC, a system LSI, a super LSI, and an ultra LSI depending on an integration degree. In the above-described RAM, a computer program for realizing the operation similar to that of each of the above-described devices is stored. The above-described microprocessor operates according to the above-described computer program, so that the system LSI realizes its function. For example, the present invention also includes a case where a beam forming method of the present invention is stored as a program of the LSI, and this LSI is inserted in the computer to perform a predetermined program (beam forming method).

Note that the method of making integrated circuit is not limited to the LSI, but it is also possible to realize by a dedicated circuit or a general-purpose processor. It is also possible to use a field programmable gate array (FPGA) which may be programed and a reconfigurable processor capable of reconfiguring connection and setting of a circuit cell in the LSI after the LSI is manufactured.

Furthermore, if an integrated circuit technology replacing the LSI appears due to advance in semiconductor technology or another derivative technology, it is naturally possible to integrate functional blocks by using the technology.

In addition, a part or all of the functions of the ultrasonic diagnostic device according to each embodiment may be realized by a processor such as a CPU executing a program. A non-transitory computer readable recording medium in which the diagnostic method of the ultrasonic diagnostic device or a program for performing the beam forming method is recorded is also possible. It is needless to say that the program may be performed by another independent computer system by recording the program and signals on a recording medium and transporting; also, the program may be distributed via a transmission medium such as the Internet.

Each component of the ultrasonic diagnostic device according to the above-described embodiment may be realized by a programmable device such as a central processing unit (CPU), a graphics processing unit (GPU), and a processor and software. The latter configuration is a so-called general-purpose computing on graphics processing unit (GPGPU). The components may be made a single circuit part or an aggregate of a plurality of circuit parts. Also, a plurality of components may be combined to make a single circuit part or an aggregate of a plurality of circuit parts.

In the ultrasonic diagnostic device according to the above-described embodiment, the ultrasonic diagnostic device includes the data storage unit as the storage device, but the storage device is not limited thereto, and it is also possible to configure such that a semiconductor memory, a hard disk drive, an optical disk drive, a magnetic storage device and the like are externally connected to the ultrasonic diagnostic device.

Division of the functional blocks in the block diagram is merely an example, and a plurality of functional blocks may be realized as one functional block, one functional block may be divided into plural, or some functions may be transferred to another functional block. Also, single hardware or software may process the functions of a plurality of functional blocks having similar functions in parallel or in time division.

Also, order in which the above-described steps are executed is illustrative to specifically describe the present invention, and may be the order other than that described above. Also, a part of the above-described steps may be executed simultaneously (in parallel) with other steps.

Also, it is configured such that the probe and the display unit are externally connected to the ultrasonic diagnostic device, but they may also be integrally provided in the ultrasonic diagnostic device.

Also, in the above-described embodiment, the probe has a probe configuration in which a plurality of piezoelectric elements is arranged in a one-dimensional direction. However, the configuration of the probe is not limited to this; for example, it is possible to use two-dimensional array transducers in which a plurality of piezoelectric transducer elements is arranged in a two-dimensional direction or a swing probe to obtain a three-dimensional tomographic image by mechanically swinging a plurality of transducers arranged in a one-dimensional direction, and they may be appropriately used depending on measurement. For example, when the two-dimensionally arranged probe is used, an irradiation position and direction of the ultrasonic beam to be transmitted may be controlled by individually changing the timing of applying the voltage to the piezoelectric transducer element and the voltage value.

In addition, the probe may include a part of the functions of the transmission/receiver in the probe. For example, on the basis of a control signal for generating a transmission electric signal output from the transmission/receiver, the transmission electric signal is generated in the probe, and the transmission electric signal is converted into the ultrasonic wave. In addition, it is possible to adopt a configuration of converting the received reflected ultrasonic wave into a reception electric signal and generating a reception signal on the basis of the reception electric signal in the probe.

In addition, at least a part of the functions of the ultrasonic diagnostic device according to each embodiment and the variation thereof may be combined. Furthermore, the numbers used above are all illustrative for specifically describing the present invention, and the present invention is not limited to the illustrative numbers.

Furthermore, the present invention also includes various variations with modifications to the extent that those skilled in the art may conceive of this embodiment.

### SUMMARY

(1) An ultrasonic signal processing device according to an embodiment is an ultrasonic signal processing device that performs speed analysis by a color flow mapping method by executing ultrasonic transmission/reception to/from a subject by driving a plurality of transducers arranged in array on an ultrasonic probe, the device provided with a transmitter that repeatedly executes a process of selecting two or more transmission conditions in predetermined order and transmitting an ultrasonic wave prescribed under a selected transmission condition into the subject at predetermined time intervals, a reception beam former that generates an acoustic line signal on the basis of a reflected ultrasonic wave received by the transducers in synchronization with the ultrasonic transmission by the transmitter, a quadrature detector that performs quadrature detection on the acoustic line signal to generate a complex acoustic line signal, and a speed calculator that performs a process of grouping a plurality of complex acoustic line signals corresponding to the same transmission condition as a packet for each transmission condition and analyzes per packet to generate speed information in the subject.

Also, an ultrasonic signal processing method according to an embodiment is an ultrasonic signal processing method that performs speed analysis by a color flow mapping method by executing ultrasonic transmission/reception to/from a subject by driving a plurality of transducers arranged in array on an ultrasonic probe provided with repeatedly executing a process of selecting two or more transmission conditions in predetermined order and transmitting an ultrasonic wave prescribed under a selected transmission condition into the subject at predetermined time intervals, generating an acoustic line signal on the basis of a reflected ultrasonic wave received by the transducers in synchronization with the process of transmitting the ultrasonic wave, performing quadrature detection on the acoustic line signal to generate a complex acoustic line signal, and performing a process of grouping a plurality of complex acoustic line signals corresponding to the same transmission condition as a packet for each transmission condition and analyzing per packet to generate speed information in the subject.

According to the ultrasonic signal processing device, the ultrasonic signal processing method, and the ultrasonic diagnostic device using the same according to an aspect of the present invention, it is possible to generate the speed information on the basis of the speed information obtained by analyzing for each transmission condition by using a plurality of transmission conditions. Therefore, it is possible to suppress variation in accuracy of the speed information caused by a fact that an optimum transmission condition differs depending on a position of an observation point, thereby improving a quality of color Doppler images of an entire target area.

(2) Also, in the ultrasonic signal processing device according to above-described (1) the speed information may include one or more of speed information, power information, and variance information at each observation point in the subject.

With the above-described configuration, it is possible to create color flow information such as a color Doppler image and a power Doppler image, and a user may check speed distribution as a color image.

(3) Also, in the ultrasonic signal processing device according to above-described (1) or (2), one of the transmission conditions may be different from the other transmission conditions in at least one of a frequency of the ultrasonic wave, a travel direction of the ultrasonic wave with respect to a transducer array, a depth of a transmission focal point at which the ultrasonic wave is focused, and a wave number of the ultrasonic wave.

With the above configuration, it is possible to transmit and receive ultrasonic waves under a suitable transmission condition to and from any observation point, suppress variation in accuracy of the speed information between the observation points, and improve the quality of the color Doppler image of the entire target area.

(4) In addition, in the ultrasonic signal processing device according to above-described (3), one of the transmission conditions may be different from the other transmission conditions in at least two of the frequency of the ultrasonic wave, the travel direction of the ultrasonic wave with respect to the transducer array, the depth of the transmission focal point at which the ultrasonic wave is focused, and the wave number of the ultrasonic wave.

With the above configuration, it is possible to use a combination of parameters suitable for each of the positions of the observation points regarding the transmission condition, so that it is possible to improve the accuracy due to difference in transmission conditions without significantly increasing the number of transmission conditions.

(5) Also, in the ultrasonic signal processing device according to above-described (1) to (4), the reception beam former may generate acoustic line signals for two or more linear areas in synchronization with the ultrasonic transmission by the transmitter.

With the above configuration, it is possible to improve spatial density of the area where the acoustic line signal is obtained.

(6) Also, in the ultrasonic signal processing device according to above-described (1) to (5), the transmitter may move a transmission transducer array used for the ultrasonic transmission by a predetermined distance in a direction in which the transducers are arranged each time the ultrasonic transmission is performed, and the reception beam former may move an area in the subject which is a target of generating the acoustic line signal by the predetermined distance in the direction in which the transducers are arranged in synchronization with the ultrasonic transmission by the transmitter.

With the above configuration, since the acoustic line signal may be generated on the basis of the reflected ultrasonic wave from the area through which the transmitted ultrasonic wave mainly passes, it is possible to improve the S/N ratio and spatial resolution of the acoustic line signal.

(7) Also, in the ultrasonic signal processing device according to above-described (6), the predetermined distance may be equal to or shorter than a width of the area in the subject which is the target of generating the acoustic line signal in the direction in which the transducers are arranged.

With the above configuration, it is possible to suppress the density at the observation point for which speed calculation is performed from becoming inhomogeneous.

(8) Also, in the ultrasonic signal processing device according to above (7), the predetermined distance may be equal to or shorter than a value obtained by multiplying the predetermined number of times by the width of the area in the subject which is the target of generating the acoustic line signal in the direction in which the transducers are arranged and dividing by the number of the transmission conditions.

With the above configuration, since the density in the direction in which the transducers are aligned at the observation point at which the speed calculation is performed is constant among the transmission conditions, it is possible to prevent unevenness in the detection accuracy of the speed between the observation points.

(9) Also, the ultrasonic signal processing device according to above-described (1) to (8), the speed calculator may calculate speed information in the subject generated by performing analysis for each packet as partial speed information, and synthesize a plurality of pieces of partial speed information to generate speed information in the subject.

With the above configuration, it is possible to obtain a compound effect due to the different transmission conditions while suppressing deterioration in accuracy caused by using the acoustic line signals of different transmission conditions as ensemble.

(10) Also, in the ultrasonic signal processing device according to above-described (9), the speed calculator may synthesize speed information in the subject by performing weighting addition of a plurality of pieces of partial speed information.

With the above configuration, it becomes possible to obtain the speed information on the basis of the partial speed information related to the transmission condition suitable for the observation point.

(11) Also, in the ultrasonic signal processing device according to above-described (9) or (10), one of the transmission conditions may be different from the other transmission conditions in a travel direction of the ultrasonic wave with respect to a transducer array, and the speed calculator may make speed information in the subject calculated for each transmission condition a vector in the same direction as the travel direction of the ultrasonic wave for each observation point within the subject, and synthesize vectors to obtain speed information at the observation point.

With the above configuration, it is possible to calculate the speed at the observation point with higher accuracy when the travel direction of ultrasonic waves differs between the transmission conditions.

(12) Also, the ultrasonic diagnostic device according to the embodiment may be provided with the ultrasonic signal processing device according to above-described (1) to (11).

By doing so, it is possible to realize the ultrasonic diagnostic device having the above characteristics.

The ultrasonic signal processing device, the ultrasonic signal processing method, and the ultrasonic diagnostic device according to the present disclosure are useful as a color Doppler image generating device and a power Doppler image generating device which improve the accuracy of the speed in the entire target area uniformly while improving the performance of the conventional ultrasonic diagnostic device and especially suppressing the decrease in frame rate.

Although embodiments of the present invention have been described and illustrated in detail, the disclosed embodiments are made for purposes of illustration and example only and not limitation. The scope of the present invention should be interpreted by terms of the appended claims

What is claimed is:

1. An ultrasonic signal processing device that performs speed analysis by a color flow mapping method by executing ultrasonic transmission/reception to/from a subject by driving a plurality of transducers arranged in array on an ultrasonic probe, the ultrasonic signal processing device comprising:
a transmitter that repeatedly executes a process of selecting two or more transmission conditions in predetermined order and transmitting an ultrasonic wave prescribed under the selected transmission condition into the subject at predetermined time intervals;
a reception beam former that generates an acoustic line signal on the basis of a reflected ultrasonic wave received by the transducers in synchronization with the ultrasonic transmission by the transmitter;
a quadrature detector that performs quadrature detection on the acoustic line signal to generate a complex acoustic line signal; and
a speed calculator that performs a process of grouping a plurality of complex acoustic line signals corresponding to the same transmission condition as a packet for each transmission condition and analyzes per packet to generate speed information in the subject.

2. The ultrasonic signal processing device according to claim 1, wherein
the speed information includes one or more of speed information, power information, and variance information at each observation point in the subject.

3. The ultrasonic signal processing device according to claim 1, wherein
one of the transmission conditions is different from the other transmission conditions in at least one of a frequency of the ultrasonic wave, a travel direction of the ultrasonic wave with respect to a transducer array, a depth of a transmission focal point at which the ultrasonic wave is focused, and a wave number of the ultrasonic wave.

4. The ultrasonic signal processing device according to claim 3, wherein
one of the transmission conditions is different from the other transmission conditions in at least two of the frequency of the ultrasonic wave, the travel direction of the ultrasonic wave with respect to the transducer array, the depth of the transmission focal point at which the ultrasonic wave is focused, and the wave number of the ultrasonic wave.

5. The ultrasonic signal processing device according to claim 1, wherein
the reception beam former generates acoustic line signals for two or more linear areas in synchronization with the ultrasonic transmission by the transmitter.

6. The ultrasonic signal processing device according to claim 1, wherein
the transmitter moves a transmission transducer array used for the ultrasonic transmission by a predetermined distance in a direction in which the transducers are arranged each time the ultrasonic transmission is performed a predetermined number of times, and
the reception beam former moves an area in the subject which is a target of generating the acoustic line signal by the predetermined distance in the direction in which the transducers are arranged in synchronization with a position of the transmission transducer array.

7. The ultrasonic signal processing device according to claim 6, wherein
the predetermined distance is equal to or shorter than a width of the area in the subject which is the target of generating the acoustic line signal in the direction in which the transducers are arranged.

8. The ultrasonic signal processing device according to claim 7, wherein
the predetermined distance is equal to or shorter than a value obtained by multiplying the predetermined number of times by the width of the area in the subject which is the target of generating the acoustic line signal in the direction in which the transducers are arranged and dividing by the number of the transmission conditions.

9. The ultrasonic signal processing device according to claim 1, wherein
the speed calculator calculates speed information in the subject generated by performing analysis for each packet as partial speed information, and synthesizes a plurality of pieces of partial speed information to generate speed information in the subject.

10. The ultrasonic signal processing device according to claim 8, wherein
the speed calculator synthesizes speed information in the subject by performing weighting addition of a plurality of pieces of partial speed information.

11. The ultrasonic signal processing device according to claim 9, wherein
one of the transmission conditions is different from the other transmission conditions in a travel direction of the ultrasonic wave with respect to a transducer array, and
the speed calculator makes speed information in the subject calculated for each transmission condition a vector in the same direction as the travel direction of the ultrasonic wave for each observation point within the subject, and synthesizes the vectors to obtain speed information at the observation point.

12. An ultrasonic diagnostic device comprising
the ultrasonic signal processing device according to claim 1 to which the ultrasonic probe is connectable.

13. An ultrasonic signal processing method that performs speed analysis by a color flow mapping method by executing ultrasonic transmission/reception to/from a subject by driving a plurality of transducers arranged in array on an ultrasonic probe, the ultrasonic signal processing method comprising:
repeatedly executing a process of selecting two or more transmission conditions in predetermined order and transmitting an ultrasonic wave prescribed under a selected transmission condition into the subject at predetermined time intervals;
generating an acoustic line signal on the basis of a reflected ultrasonic wave received by the transducers in synchronization with the process of transmitting the ultrasonic wave;
performing quadrature detection on the acoustic line signal to generate a complex acoustic line signal; and
performing a process of grouping a plurality of complex acoustic line signals corresponding to the same transmission condition as a packet for each transmission condition and analyzing per packet to generate speed information in the subject.
